(19) United States
(12) Patent Application Publication
(10) Pub. No.: US 2026/0335042 A1
(43) Pub. Date: Oct. 29, 2026

(54) Apparatus and Method for Washing and Drying Medical Devices

(71) Applicant: LiViliti Health Products, Lake City, FL (US)

(72) Inventor: Brian Sharpe, Irmo, SC (US)

(73) Assignee: LiViliti Health Products, Lake City, FL (US)

(21) Appl. No.: 19/578,166

(22) Filed: Mar. 25, 2026

Related U.S. Application Data
(60) Provisional application No. 63/793,422, filed on Apr. 23, 2025.

(51) Int. Cl. A61B 90/70; B08B 3/04; F26B 21/00

(52) U.S. Cl. CPC A61B 90/70; B08B 3/04; F26B 21/006

(57) ABSTRACT

An apparatus for washing and drying a medical device, including a base including a blower, and a wash basin lid removably mounted to the apparatus base for movement between (a) a removed position in which the wash basin lid is removed from the apparatus base and arranged in a face-up orientation and (b) a mounted position in which the wash basin lid is mounted to the apparatus base and arranged in a face-down orientation. In the removed position, the wash basin lid defines a washing compartment to contain a volume of liquid for washing the medical device, and in the mounted position, the wash basin lid and base define a drying compartment for drying the medical device. The blower is configured to generate an air flow in the drying compartment to facilitate drying of the medical device

### RELATED PATENT APPLICATION

This application claims priority to commonly owned United States Provisional Patent Application No. 63/793,422 filed Apr. 23, 2025, the entire contents of which are hereby incorporated by reference for all purposes.

### TECHNICAL FIELD

The present invention relates to an apparatus and method for washing and drying medical devices, for example CPAP accessories.

### BACKGROUND

Many medical devices, for example CPAP accessories (e.g., masks, nasal pillows, hoses, etc.), require or benefit from regular cleaning, for example to remove bacteria and/or other contaminants that may cause infection, illness, or irritation of the wearer. In addition, it is often important to dry a washed medical device, for example to prevent the growth of mildew or bacteria. Accordingly, there is a need for an apparatus for both washing and drying of medical devices, e.g., CPAP accessories.

### SUMMARY

A wash/dry apparatus and method are disclosed for washing and drying medical device(s), e.g., CPAP accessories. The wash/dry apparatus may include an apparatus base and a removable wash basin that functions as both a wash basin for washing the medical device(s), and a cover mountable on the apparatus base to define the drying compartment for drying the washed medical device(s).

One aspect provides an apparatus for washing and drying a medical device, including an apparatus base including a first blower, and a removable a wash basin lid including a lid base and at least one lid sidewall extending from the lid base. The wash basin lid may be removably mounted to the apparatus base and movable between (a) a mounted position in which the wash basin lid is mounted to the apparatus base in a face-down orientation, and (b) a removed position in which the wash basin lid is removed from the apparatus base and arranged in a face-up orientation. In the removed position, the wash basin lid may define a water-tight washing compartment to contain a volume of liquid for washing the medical device. In the mounted position, the wash basin lid and the apparatus base may define a drying compartment between the wash basin lid and the apparatus base for drying the medical device. The first blower may be connected to the drying compartment and configured to generate a first air flow in the drying compartment to facilitate drying of the medical device.

In some examples, the apparatus base includes a hose port configured for removable attachment of a medical hose element, and a second blower connected to the hose port to generate a second air flow through the hose port.

In some examples, the apparatus base includes an air flow outlet opening into the drying compartment, the blower is connected to the air flow outlet to generate the first air flow through the air flow outlet opening and into the drying compartment, and the hose port is spaced apart from the air flow outlet.

In some examples, the apparatus base includes an air flow outlet opening into the drying compartment, and a hose port configured for removable attachment of a medical hose element, and the blower is configured to generate (a) the first air flow through the air flow outlet opening and into the drying compartment and (b) a second air flow through the hose port.

In some examples, the wash basin lid includes at least one vent opening in the at least one sidewall to vent air from the drying compartment in the mounted position of the wash basin lid.

In some examples, the wash basin lid includes a plurality of lid feet extending from the lid base or from the at least one lid sidewall, the plurality of lid feet holding the lid base spaced apart from an underlying support surface in the removed position of the wash basin lid.

In some examples, the at least one lid sidewall includes a first sidewall having a first vertical height, and a second lid sidewall having a second vertical height greater than the first vertical height, in the removed position of the wash basin lid, and the second sidewall includes at least one vent opening to vent air from the drying compartment in the mounted position of the wash basin lid.

In some examples, the water-tight washing compartment defined by the wash basin lid is shaped to contain a volume of liquid of at least one gallon.

In some examples, the water-tight washing compartment defined by the wash basin lid is shaped to contain a volume of liquid of at least 1.5 gallons.

In some examples, the apparatus includes a tray removably mounted in the drying compartment to support the medical device.

Another aspect provides an apparatus for washing and drying a medical device, the apparatus including an apparatus base and a wash basin lid removably mounted to the apparatus base. The apparatus base may includes a blower to generate a first air flow, control electronics to control the blower, and at least one lid mounting structure. In a removed position of the wash basin lid, the wash basin lid defines a water-tight washing compartment to contain a volume of liquid for washing the medical device. In a mounted position of the wash basin lid, the wash basin lid is mounted to the at least one lid mounting structure of the apparatus base, wherein a drying compartment is defined between the apparatus base and the mounted wash basin lid for drying the medical device, and wherein the first blower is connected to the drying compartment and controllable by the control electronics to generate an air flow in the drying compartment to facilitate drying of the medical device.

In some examples, the apparatus base includes a hose port configured for removable attachment of a medical hose element, and wherein the blower is connected to the hose port to generate air flow through the hose port.

In some examples, the apparatus base includes a hose port configured for removable attachment of a medical hose element, and an additional blower connected to the hose port to generate air flow through the hose port.

In some examples, the wash basin lid includes a lid base and at least one lid sidewall extending from the lid base, wherein at least one lid sidewall includes at least one vent opening to vent air from the drying compartment, in the mounted position of the wash basin lid.

Another aspect provides a method for washing and drying a medical device using an apparatus including an apparatus base including a wash basin lid removably mountable to the apparatus base. The wash basin lid is arranged in a face-up orientation removed from the apparatus base, the wash basin lid including a lid base and at least one lid sidewall extending from the lid base defining a water-tight washing compartment. The medical device is arranged in the water-tight washing compartment and at least partially filling the water-tight washing compartment with a liquid. The liquid is used to wash the medical device. After washing the medical device, the medical device is arranged on or supported by the apparatus base. The wash basin lid is mounted to the apparatus base in a face-down orientation, such that the medical device is located in a drying compartment defined between the wash basin lid and apparatus base. A blower in the apparatus base is operated to generate a first air flow in the drying compartment to facilitate drying of the medical device.

In some examples, the medical device comprises a medical hose element, arranging the medical device on or supported by the apparatus base comprises attaching the medical hose element to a hose port provided in the apparatus base, and operating the first blower to generate the first flow in the drying compartment comprises operating the first blower to generate the first flow through the hose port and into an interior of the medical hose element.

In some examples, the method includes operating the first blower to simultaneously generate (a) the first air flow through into the drying compartment to facilitate drying of the medical device and (b) a second air flow through a hose port provided in the apparatus base to facilitate drying of a medical hose element attached to the hose port.

In some examples, the method includes attaching a medical hose element to a hose port provided in the apparatus base, and operating a second blower provided in the apparatus base to generate a second air flow through the hose port and into an interior of the medical hose element.

In some examples, arranging the medical device on or supported by the apparatus base comprises arranging the medical device on a removable tray supported by the apparatus base.

### BRIEF DESCRIPTION OF THE DRAWINGS

Example aspects of the present disclosure are described below in conjunction with the figures, in which:

FIGS. 1 and 2 are three-dimensional front and rear views, respectively, of an example wash/dry apparatus in an assembled state;

FIG. 3 shows the example wash basin lid in a removed position and face-up orientation, e.g., for washing at least one medical device;

FIGS. 4A and 4B show two example implementations of an apparatus base of the example wash/dry apparatus shown in FIGS. 1 and 2, with the wash basin lid removed; and

FIG. 5 shows a flowchart of an example method of washing and drying one or more medical device using an example wash/dry apparatus as shown in FIGS. 1-4B.

It should be understood the reference number for any illustrated element that appears in multiple different figures has the same meaning across the multiple figures, and the mention or discussion herein of any illustrated element in the context of any particular figure also applies to each other figure, if any, in which that same illustrated element is shown.

### DETAILED DESCRIPTION

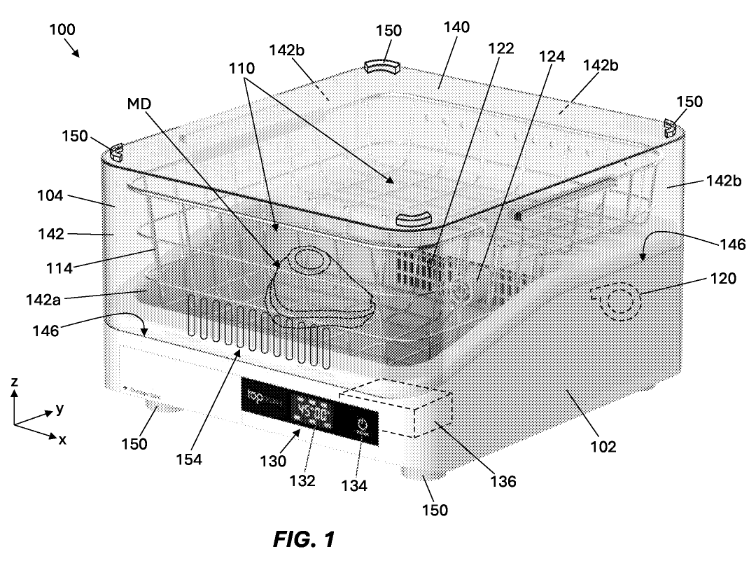

Embodiments of the present invention provide an apparatus for washing and drying medical devices, e.g., CPAP accessories. FIGS. 1-4B shows one example wash/dry apparatus 100 for washing and drying medical device(s).

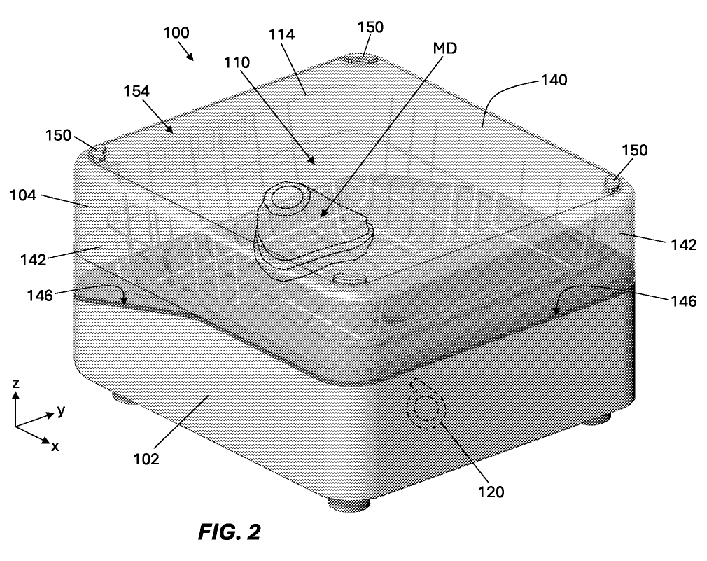

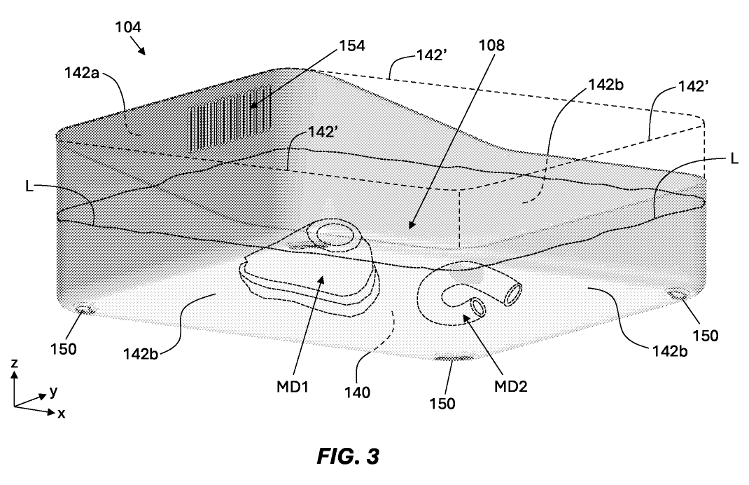

FIGS. 1 and 2 are three-dimensional front and rear views, respectively, of the example wash/dry apparatus 100 in an assembled state. The example wash/dry apparatus 100 may include (a) an apparatus base 102 and (b) a wash basin lid 104 removably mounted to the apparatus base 100 for movement between (i) a removed position (shown in FIG. 3 discussed below) in which the wash basin lid 104 is removed from the apparatus base 102 and arranged in a face-up orientation for washing medical device(s) and (ii) a mounted and face-down position (shown in FIGS. 1-2) in which the wash basin lid 104 is mounted to the apparatus base 102 in a face-down orientation for drying the medical device(s). In the removed and face-up position (shown in FIG. 3 discussed below), the wash basin lid 104 defines a water-tight washing compartment 108 to contain a volume of liquid for washing the medical device. In the mounted and face-down position, the wash basin lid 104 and apparatus base 102 define a drying compartment 110 for drying the medical device(s). Thus, the wash basin lid 104 may function as both (a) a wash basin for washing the medical device(s) and (b) a cover for the wash/dry apparatus 100 to define the drying compartment 110 for drying the medical device(s). FIGS. 1 and 2 show an example medical device MD, in particular a CPAP mask component, arranged in the drying compartment 110.

The wash/dry apparatus 100 may include a tray 114 removably arranged on the apparatus base 102 to hold or otherwise support medical device(s) during the drying process, and allow water to drip through the bottom of the tray 114 and into a drain 116 (shown in FIGS. 3A and 3B) formed in the apparatus base 102.

The apparatus base 102 may include at least one blower 120 connected to the drying compartment 110 to generate at least one air flow in the drying compartment 110 to facilitate drying of medical device(s) in the drying compartment 110. The apparatus base 102 may include an air flow outlet opening 122 opening into the drying compartment 110, and (optionally) a hose port 124 configured for removable attachment of a medical device (e.g., a medical hose element) to be dried. Each blower 120 may include a blower motor and fan to generate a respective air flow for drying respective medical device(s).

In some examples, e.g., as discussed below with reference to FIG. 3A, the apparatus base 102 may include a blower 120 configured to generate (a) a first air flow through the air flow outlet opening 122 and into the drying compartment 110 for drying medical device(s) arranged on the tray 114 and (b) a second air flow through a hose port 124 for drying a medical device (e.g., a medical hose element) attached to the hose port 124. The hose port 124 may be arranged within the boundary of an air flow outlet opening 122 (e.g., as shown in FIGS. 1 and 3A), or alternatively may be spaced apart from an air flow outlet opening 122 (e.g., as shown in FIG. 3B).

In other examples, e.g., as discussed below with reference to FIG. 3B, the apparatus base 102 may include (a) a first blower 120a to generate a first air flow through an air flow outlet opening 122 and into the drying compartment 110 for drying medical device(s) arranged on the tray 114, and (b) a separate second blower 120b to generate a second air flow through a hose port 124 for drying a medical device (e.g., a medical hose element) attached to the hose port 124.

The apparatus base 102 may include a user interface 130 allowing a user to operate the wash/dry apparatus 100, for example by controlling blower(s) 120. The user interface 130 may include a display 132 and input elements 134. The display 132 may include, for example, an LCD screen, an array of LED elements, or other type(s) of display device(s) configured to display operational settings, operational status (e.g., a timer), and/or other information related to the wash/dry apparatus 100. Input elements 134 may include, for example, physical or virtual buttons, switches, sliders, etc. In some examples, the user interface 130 may include touch screen including input elements 134 integrated with a display 132.

The apparatus base 102 may further include control electronics 136 for controlling the blower(s) 120, e.g., including a processor and logic instructions (e.g., embodied in firmware and/or software) stored in memory and executable by the processor to perform automated functions of the apparatus, including controlling the blower motor. In some examples, the control electronics 136 may define one or more drying processes having predefined blower run times and/or blower speed/power settings. In addition or alternatively, the control electronics 136 may allow the user to program a desired blower run time and/or blower speed/power via respective input elements 134.

The wash basin lid 104 may include a lid base 140 and at least one lid sidewall 142 (including lid sidewalls 142a and 142b shown in FIGS. 1-4B) extending from the lid base 140, collectively defining a water-tight washing compartment 108 (shown more clearly in FIG. 3) to contain a volume of liquid L for washing medical device(s) arranged in the water-tight washing compartment 108. A plurality of optional lid feet 150 may extend from the lid base 140, which lid feet 150 may be configured to hold the lid base spaced apart from an underlying support surface in the removed and face-up orientation of the wash basin lid 104. The lid feet 150 may provide stability for the wash basin lid 104, resist sliding of the wash basin lid 104 on a hard surface (e.g., a countertop), and help keep the wash basin lid 104 clean. In some examples, the lid feet 105 may comprise silicon or rubber pads (e.g., having a thickness in the range of 5-20mm) affixed to the wash basin lid 104 (e.g., using adhesive). In other examples, lid feet 105 may be formed (e.g., molded) integrally with the wash basin lid 104.

In some examples, the lid base 140 is visibly distinct from the lid sidewalls 142, for example where planar or generally planar lid sidewalls 142 extend orthogonally from planar or generally planar lid base 140, as in the example shown in FIG. 1-4B. In other examples, e.g., wherein the wash basin lid 104 has a more rounded shape, the lid base 140 may be less distinct from the lid sidewall(s) 142. In such examples, the lid base 140 may be defined as either (a) the portion of the wash basin lid 104 that rests on a flat underlying support surface (e.g., the top of a vanity, counter, or table), or (b) in an example including lid feet 150 that lift the wash basin lid 104 above an underlying support surface, the portion of the wash basin lid 104 within a perimeter defined by the lid feet 150.

The lid base 140 and lid sidewall(s) 142 may have any suitable shapes and sizes. For example, the lid base 140 may be flat (planar) or may have a curved shape (e.g., concave in the face-up orientation of the wash basin lid 104). In the example shown in FIGS. 1-4B, the wash basin lid 104 extends in an x-y plane, and four lid sidewalls 142 extend orthogonally from the lid base 140 (in the z-direction), to form a generally rectangular perimeter of the wash basin lid 104. In other examples, wash basin lid 104 may include a single lid sidewall 142 extending from a circular or rounded perimeter of the lid base 140 in the z-axis direction to define a circular or rounded perimeter of the wash basin lid 104. In other examples, the wash basin lid 104 may have a rounded bowl shape with lid feet 150 extending therefrom, wherein the lid base 140 (as discussed above) is defined as the portion of the wash basin lid 104 within a perimeter defined by the lid feet 150, and the lid sidewall 142 is defined by the remaining portion of the wash basin lid 104 extending from the lid base 140.

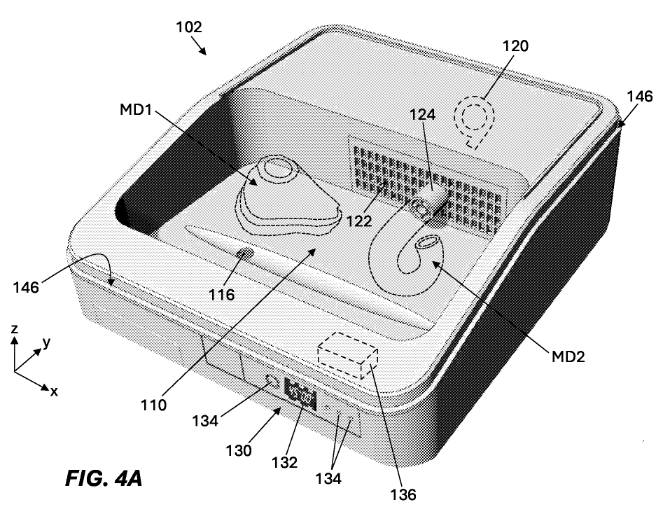

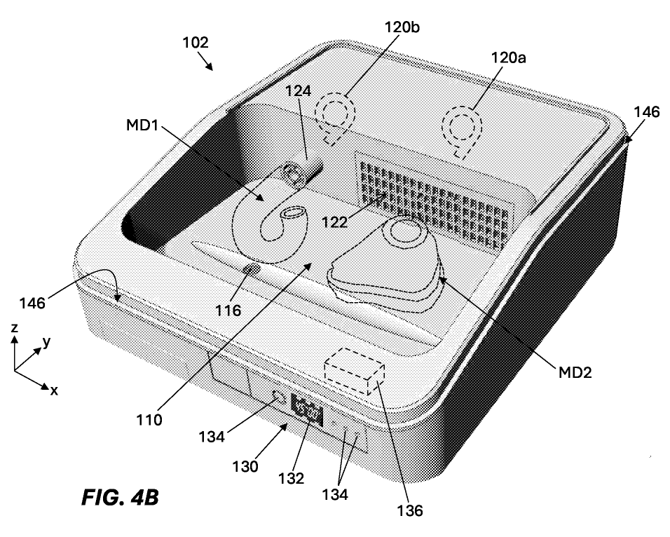

As shown in FIGS. 1 and 2, and more clearly in FIGS. 4A-4B, the apparatus base 102 may include lid mounting structure(s) 146 on which the wash basin lid 104, in particular lid sidewall(s) 142, may be mounted. The lid mounting structure(s) 146 may include one or more ledge, shoulder, ridge, lip, flange, channel, groove, clip, or other structure(s) shaped and sized to receive and align the wash basin lid 104 relative to the apparatus base 102. In some examples, the lid mounting structure(s) 146 provide a mounting surface on which the mounted wash basin lid 104 may rest, while allowing the wash basin lid 104 to be freely lifted off the apparatus base 102. In other examples, the lid mounting structure(s) 146 may additionally physically lock or otherwise secure the wash basin lid 104 to the apparatus base 102, such that the wash basin lid 104 may not be freely lifted off the apparatus base 102. For example, the lid mounting structure(s) 146 may include flexible clips or other releasable locking elements.

At least one lid sidewall 142 may include at least one vent opening 154 to vent air from the drying compartment 110 defined between the wash basin lid 104 and apparatus base 102, to thereby allow air flow (generated by blower(s) 120) through and out of the drying compartment 110 into the surrounding environment, e.g., to facilitate drying of medical device(s) MD in the drying compartment 110.

FIG. 3 shows the example wash basin lid 104 in the removed position and face-up orientation, e.g., for washing at least one medical device MD. As shown, the example wash basin lid 104 includes a lid base 140 and lid sidewalls 142 (including sidewalls 142a and 142b) that define a water-tight washing compartment 108 to contain a volume of liquid L (e.g., a water and soap solution) for washing at least one medical device (in the illustrated example, medical devices MD1 and MD2). In some examples, the water-tight washing compartment 108 may contain a volume of liquid of at least one gallon, or at least 1.5 gallons, or at least 2 gallons.

In the illustrated example, a first sidewall 142a has a greater height (in the z-direction) than the other lid sidewalls 142b, wherein vent openings 154 (for venting air from the drying compartment 110 in the mounted and face-down position of the wash basin lid. 104 shown in FIGS. 1 and 2) are formed in the first sidewall 142a at a location above the top of the water-tight washing compartment 108 defined by the shorter lid sidewalls 142b (wherein the shorter lid sidewalls 142b define the size of the water-tight washing compartment 108).

In other examples, all lid sidewalls 142 may all have the same height, e.g., as indicated by the dashed line 142’ representing four lid sidewalls having the same height.

FIGS. 4A and 4B show two example implementations of the apparatus base 102 of the example wash/dry apparatus 100 with the wash basin lid 104 removed, e.g., to illustrate example arrangement of medical device(s) to be dried in the drying compartment 110 (defined between the wash basin lid 104 and apparatus base 102 when the wash basin lid 104 is mounted to the apparatus base 102, e.g., as shown in FIG. 1 and 2).

FIG. 4A shows a first example implementation of the apparatus base 102 including (a) an air flow outlet 122 opening into the drying compartment 110, (b) a hose port 124 configured for removable attachment of a medical hose element, and (c) a blower 120 fluidically connected to the air flow outlet 122 and the hose port 124 to generate (i) a first air flow through the air flow outlet opening 122 and into the drying compartment 110 and (ii) a second air flow through the hose port 124. As shown, the air flow outlet 122 may be embodied as a grille defining an array of openings, and the hose port 124 is co-located with the air flow outlet 122 (e.g., located within a perimeter of the air flow outlet 122).

FIG. 4A shows two example medical devices supported on the apparatus base 102, in particular a CPAP mask device MD1 and a CPAP hose element MD2. The CPAP mask device MD1 and CPAP hose element MD2 may be arranged on a removable tray 114 (shown in FIGS. 1 and 2), and one end of the CPAP hose element MD2 may be attached to the hose port 124. When the wash basin lid 104 is mounted on the apparatus base 102 (thereby defining the drying compartment 110 containing the CPAP mask device MD1 and CPAP hose element MD2), the blower 120 may be operated to generate (i) the first air flow through the air flow outlet opening 122 and into the drying compartment 110, to facilitate drying of the CPAP mask device MD1 and an exterior of the CPAP hose element MD2, and (ii) the second air flow through the hose port 124 and through the CPAP hose element MD2, to facilitate drying of an interior of the CPAP hose element MD2.

Control electronics 136 may define one or more drying processes having predefined blower run times and/or blower speed/power settings for the blower 120, and may allow user programming of a blower run time and/or blower speed/power via respective input elements 134.

FIG. 4B shows a second example implementation of the apparatus base 102 including (a) an air flow outlet 122 opening into the drying compartment 110, (b) a hose port 124 configured for removable attachment of a medical hose element, (c) a first blower 120a fluidically connected to the air flow outlet 122 to generate a first air flow through the air flow outlet opening 122 and into the drying compartment 110, and (d) a second bower 120b fluidically connected to the hose port 124 to generate a second air flow through the hose port 124. As shown, the air flow outlet 122 may be embodied as a grille defining an array of openings, and the hose port 124 may be located spaced apart from the air flow outlet 122.

Like FIGS. 4A, 4B also shows two example medical devices supported on the apparatus base 102, in particular a CPAP mask device MD1 and a CPAP hose element MD2. The CPAP mask device MD1 and CPAP hose element MD2 may be arranged on a removable tray 114 (shown in FIGS. 1 and 2), and one end of the CPAP hose element MD2 may be attached to the hose port 124. When the wash basin lid 104 is mounted on the apparatus base 102 (thereby defining the drying compartment 110 containing the CPAP mask device MD1 and CPAP hose element MD2), the first blower 120a may be operated to generate the first air flow through the air flow outlet opening 122 and into the drying compartment 110, to facilitate drying of the CPAP mask device MD1 and an exterior of the CPAP hose element MD2, and the second blower 120b may be operated to generate the second air flow through the hose port 124 and through the CPAP hose element MD2, to facilitate drying of an interior of the CPAP hose element MD2.

Control electronics 136 may define one or more drying processes having predefined blower run times and/or blower speed/power settings for each blower 120a and 120b, and may allow user programming of blower run times and/or blower speed/power settings via respective input elements 134. The first blower 120a and second blower 120b may be operated concurrently, in an alternating manner, or according to any other control protocol. In some examples, first blower 120a and second blower 120b may be controlled independently (e.g., based on inputs at input elements 134), for example to activate or deactivate the second blower 120b based on whether a hose element to be dried is attached to the hose port 124.

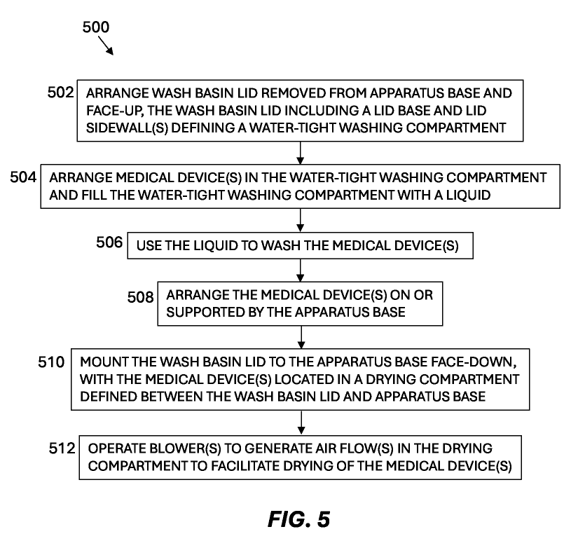

FIG. 5 shows a flowchart of an example method 500 of washing and drying one or more medical device using the example wash/dry apparatus 100 discussed above. At 502, the wash basin lid 104 is arranged in a face-up orientation removed from the apparatus base 102, e.g., as shown in FIG. 3. For example, the wash basin lid 104 may be arranged on a countertop, table, or other flat surface.

At 504, the medical device(s) are arranged in the water-tight washing compartment 108 defined by the face-up wash basin lid 104, and the water-tight washing compartment 108 is at least partially filled with cleaning liquid, e.g., a water and soap solution.

At 506, the medical device(s) may be washed using the cleaning liquid, e.g., including soaking, scrubbing, rinsing, or otherwise cleaning the medical device(s).

After washing the medical device(s), the medical device(s) may be arranged on or supported by the apparatus base 102 at 508. For example, the medical device(s) may be arranged in the removable tray 114 supported on the apparatus base 102. If the medical device(s) include a medical hose device, the medical hose device may be attached to a hose port 124.

At 510, the wash basin lid 104 may be emptied of any cleaning liquid and mounted to the apparatus base 102 in the face-down orientation, such that the medical device(s) are located in the drying compartment 110 defined between the wash basin lid 104 and apparatus base 102.

At 512, blower(s) 120 provided in the apparatus base 102 may be operated to generate air flow(s) in the drying compartment 110 to facilitate drying of the medical device(s), e.g., as discussed above regarding FIGS. 4A and 4B. Air flow in the drying compartment 110 may vent out through vent opening(s) 154, as discussed above.

Thus, the wash basin lid 104 functions as both (a) a wash basin for washing the medical device(s) and (b) a cover for the wash/dry apparatus 100 to define the drying compartment 110 for drying the medical device(s).

Although example embodiments have been described above, other variations and embodiments may be made from this disclosure without departing from the spirit and scope of these embodiments.

1. An apparatus for washing and drying a medical device, the apparatus comprising:
an apparatus base including a first blower; and
a wash basin lid including:
a lid base; and
at least one lid sidewall extending from the lid base;
wherein the wash basin lid is removably mounted to the apparatus base and movable between (a) a mounted position in which the wash basin lid is mounted to the apparatus base in a face-down orientation, and (b) a removed position in which the wash basin lid is removed from the apparatus base and arranged in a face-up orientation;
wherein in the removed position, the wash basin lid defines a water-tight washing compartment to contain a volume of liquid for washing the medical device;
wherein in the mounted position, the wash basin lid and the apparatus base define a drying compartment between the wash basin lid and the apparatus base for drying the medical device; and
wherein the first blower is connected to the drying compartment and configured to generate a first air flow in the drying compartment to facilitate drying of the medical device.

2. The apparatus of claim 1, wherein the apparatus base includes:
a hose port configured for removable attachment of a medical hose element; and
a second blower connected to the hose port to generate a second air flow through the hose port.

3. The apparatus of claim 2, wherein:
the apparatus base includes an air flow outlet opening into the drying compartment;
the blower is connected to the air flow outlet to generate the first air flow through the air flow outlet opening and into the drying compartment; and
the hose port is spaced apart from the air flow outlet.

4. The apparatus of claim 1, wherein:
the apparatus base includes:
an air flow outlet opening into the drying compartment; and
a hose port configured for removable attachment of a medical hose element; and
the blower is configured to generate (a) the first air flow through the air flow outlet opening and into the drying compartment and (b) a second air flow through the hose port.

5. The apparatus of claim 1, wherein the wash basin lid includes at least one vent opening in the at least one sidewall to vent air from the drying compartment in the mounted position of the wash basin lid.

6. The apparatus of claim 1, wherein the wash basin lid includes:
a plurality of lid feet extending from the lid base or from the at least one lid sidewall, the plurality of lid feet holding the lid base spaced apart from an underlying support surface in the removed position of the wash basin lid.

7. The apparatus of claim 1, wherein:
the at least one lid sidewall includes a first sidewall having a first vertical height, and a second lid sidewall having a second vertical height greater than the first vertical height, in the removed position of the wash basin lid; and
the second sidewall includes at least one vent opening to vent air from the drying compartment in the mounted position of the wash basin lid.

8. The apparatus of claim 1, wherein the at least one lid sidewall includes a plurality of sidewalls defining a perimeter of the wash basin lid, wherein each of the plurality of sidewalls has the same vertical height.

9. The apparatus of claim 1, wherein the water-tight washing compartment defined by the wash basin lid is shaped to contain a volume of liquid of at least one gallon.

10. The apparatus of claim 1, wherein the water-tight washing compartment defined by the wash basin lid is shaped to contain a volume of liquid of at least 1.5 gallons.

11. The apparatus of claim 1, including a tray removably mounted in the drying compartment to support the medical device.

12. An apparatus for washing and drying a medical device, the apparatus comprising:
an apparatus base including:
a blower to generate a first air flow;
control electronics to control the blower; and
at least one lid mounting structure;
a wash basin lid removably mounted to the apparatus base;
wherein in a removed position of the wash basin lid, the wash basin lid defines a water-tight washing compartment to contain a volume of liquid for washing the medical device;
wherein in a mounted position of the wash basin lid, the wash basin lid is mounted to the at least one lid mounting structure of the apparatus base, wherein a drying compartment is defined between the apparatus base and the mounted wash basin lid for drying the medical device, and wherein the first blower is connected to the drying compartment and controllable by the control electronics to generate an air flow in the drying compartment to facilitate drying of the medical device.

13. The apparatus of claim 12, wherein the apparatus base includes:
a hose port configured for removable attachment of a medical hose element; and
wherein the blower is connected to the hose port to generate air flow through the hose port.

14. The apparatus of claim 12, wherein the apparatus base includes:
a hose port configured for removable attachment of a medical hose element; and
an additional blower connected to the hose port to generate air flow through the hose port.

15. The apparatus of claim 12, wherein the wash basin lid includes a lid base and at least one lid sidewall extending from the lid base;
wherein at least one lid sidewall includes at least one vent opening to vent air from the drying compartment, in the mounted position of the wash basin lid.

16. A method for washing and drying a medical device using an apparatus including an apparatus base including a wash basin lid removably mountable to the apparatus base, the method comprising:
arranging the wash basin lid in a face-up orientation removed from the apparatus base, the wash basin lid including a lid base and at least one lid sidewall extending from the lid base defining a water-tight washing compartment;
arranging the medical device in the water-tight washing compartment and at least partially filling the water-tight washing compartment with a liquid;
using the liquid to wash the medical device;
after washing the medical device, arranging the medical device on or supported by the apparatus base;
mounting the wash basin lid to the apparatus base in a face-down orientation, such that the medical device is located in a drying compartment defined between the wash basin lid and apparatus base;
operating a first blower provided in the apparatus base to generate a first air flow in the drying compartment to facilitate drying of the medical device.

17. The method of claim 16, wherein:
the medical device comprises a medical hose element;
arranging the medical device on or supported by the apparatus base comprises attaching the medical hose element to a hose port provided in the apparatus base;
operating the first blower to generate the first flow in the drying compartment comprises operating the first blower to generate the first flow through the hose port and into an interior of the medical hose element.

18. The method of claim 16, comprising operating the first blower to simultaneously generate (a) the first air flow through into the drying compartment to facilitate drying of the medical device and (b) a second air flow through a hose port provided in the apparatus base to facilitate drying of a medical hose element attached to the hose port.

19. The method of claim 16, comprising:
attaching a medical hose element to a hose port provided in the apparatus base; and
operating a second blower provided in the apparatus base to generate a second air flow through the hose port and into an interior of the medical hose element.

20. The method of claim 16, wherein arranging the medical device on or supported by the apparatus base comprises arranging the medical device on a removable tray supported by the apparatus base.
